(19) United States
(12) Patent Application Publication
(10) Pub. No.: US 2023/0323292 A1
(43) Pub. Date: Oct. 12, 2023

(54) METHODS FOR PRODUCING A (THREE DIMENSIONAL) NEURAL TISSUE

(71) Applicant: TECHNISCHE UNIVERSITEIT EINDHOVEN, EINDHOVEN (NL)

(72) Inventors: Aref SABERI, EINDHOVEN (NL); Nicholas Agung KURNIAWAN, EINDHOVEN (NL); Carolina Victoria Catharina BOUTEN, EINDHOVEN (NL)

(21) Appl. No.: 18/009,096

(22) PCT Filed: Jun. 8, 2021
(86) PCT No.: PCT/NL2021/050363

§ 371 Date: Dec. 8, 2022

(30) Foreign Application Priority Data
Jun. 8, 2020 (NL) 2025784

(51) Int. Cl. C12N 5/0793

(52) U.S. Cl. CPC C12N 5/0619; C12N 2513/00; C12N 2506/45; C12N 2501/115; C12N 2501/119; C12N 2509/00; C12N 2533/90

(57) ABSTRACT

The method relates to an in vitro method of producing a (three dimensional) neural tissue composition, the method comprising the steps of re-suspending cells that are obtained by culturing pluripotent stem cells in a neural induction medium in cell culture substrate and culturing said resuspended cells in the presence of an neural differentiation medium.

### INTRODUCTION

Various characteristics of a developing central nervous system, including sensory input-output, neuronal migration and regionalization have been recapitulated through recent advances in the culture of brain organoids. These organoids can model the fundamental processes in brain development and disease. A remaining critical challenge, however, is to achieve complex neuronal networks with functional interconnectivity as in native brain tissue. Generation of current organoid models originates from classic dissociation-reaggregation paradigms, often relying on mechanically-enforced quick reaggregation of pluripotent stem cells.

In light of this, new methods for producing a (three dimensional) neural tissue composition would be highly desirable. In particular, there is a need in the art for reliable, efficient and reproducible methods to provide (three dimensional) neural tissue composition that allow to be used in, for example, the study of cerebral tissue, in particular in the context of neurological disorders. Accordingly, a technical problem underlying the present invention can be seen in the provision of such methods and uses for complying with any of the aforementioned needs. The technical problem is solved by the embodiments characterized in the claims and herein below.

This introduction includes information that may be useful in understanding the present invention. It is not an admission that any of the information provided herein is prior art or relevant to the presently claimed invention, or that any publication specifically or implicitly referenced is prior art.

### FIGURES

FIG. 1. Formation of human cerebral tissues via MARC. (a) Schematics of the MARC culture method, showing the different culture steps. Timeline and additives supplied in each step are indicated. (b) Example phase-contrast images at the different 3D-culture phases, showing the matrix-supported active reaggregation of the cells into cerebral tissues. Single dissociated cells suspended in Matrigel grew into small spheroids (Day 1-7). During pre-terminal differentiation, neurite outgrowths extended from the spheroids (white arrows) and merged into neurite bundles (white arrowheads) between spheroids (Day 10, 15). The spheroids migrated using these neurite bundles and merged into large cerebral tissues (Day 20). Scale bar: 500 μm. (c) Immunohistochemical staining of cerebral tissues at Day 90 revealed the presence markers of neural progenitor cells (NPCs; PAX6), early and mature neurons (Tuj1 and MAP2), mature GABAergic neurons (excitatory VGLUT1 and inhibitory VGAT transporters), mature dopaminergic neurons (DAT), astrocytes (GFAP), and oligodendrocytes (Olig2) were expressed (red), indicating multiregional cerebral tissues with extensive cellular diversity. Blue=DAPI. Scale bars: 1 mm.

FIG. 2. Functional network interconnectivity in intact MARC-produced cerebral tissues. (a) Neuronal activity in a cerebral tissue at week 4 of MARC culture. A snapshot of live fluorescence calcium imaging on the intact cerebral tissue is overlaid with regions-of-interest (ROIs) color-coded based on the frequency of detected calcium surges (“activity”) in each ROI. Scale bar: 100 μm. (b) Representative time traces of normalized intensity (ΔF/F) from 20 ROIs. Blue crosses indicate detected transient spikes. Time traces from 387 ROIs in the image are used to compute the correlation coefficient rᵢⱼ between ROI pairs i and j (see Methods for details of calculation). (c) Correlation matrix between any pair of the 387 ROIs, showing the r-value for each pair. (d) Depiction of functional connectivity network in the cerebral tissue. The contour of the cerebral tissue, corresponding to a, is shown. Two ROIs are defined to be functionally connected when r>0.6 and shown as gray lines in the connectivity map. Further, each ROI in the connectivity map is color coded based on the number of functional connections it has. (e) The same correlation matrix as in c, but with the ROIs sorted based on the number of functional connections they have. The high density of ROI pairs with high number of connections and high r-value suggests a non-random network topology. (f) Distribution of the number of functional connections. The distribution follows a power law with a decay power of −2, demonstrating a scale-free cerebral tissue functional network. (g) Clustered correlation matrix. To test whether the network exhibits modular topology, the functional connectivities are analyzed using Louvain algorithm¹⁹, which indicates that the network contains three communities/modules. The correlation matrix in d is then reordered so that the nodes in the same module (color coded with 1, 2, and 3) is positioned together. The high density of cross-module node pairs with high r-values (arrows) suggests the existence of hub connections between modules. (h) The localization of the nodes in the 3 modules. The color of the nodes correspond to the color coding of the 3 modules in g. The inset shows the spatial regions that enclose the nodes identified in the 3 modules, together with the hub nodes (defined as nodes with number of intra-module connections larger than 90^{th} percentile in the module) and the cross-module hub connections (gray lines). (i) Topological representation of the intra-module functional connectivity networks. To illustrate the topological proximity of highly connected nodes, each module network is shown using Fruchterman-Reingold algorithm²² where the length of the lines connecting nodes is proportional to 1-r (i.e. short lines indicate high correlation coefficient between the node pairs, and the converse). The hubs in each module are also indicated. The central positioning of the hub nodes, as well as the close topological proximity between the hub nodes, highlight their status as intramodular connector nodes in the cerebral tissue functional network.

FIG. 3. Formation and interconnection of MARC-produced cerebral tissues in the iS3CC chip. (a) The design and features of the iS3CC chip. A schematic illustration of the iS3CC chip (i) and a cross-section view of the iS3CC chip including the features: the PDMS body, chambers wherein the cerebral tissues are cultured, the porous membrane, and the glass slide (ii). (b) A photograph of an assembled iS3CC device where chambers are filled with red and blue dyes (left and right chamber). (c) Side-view schematics of the progress of MARC culture in the iS3CC chip, resulting in interconnected cerebral tissues. Bottom-view phase-contrast images of both chambers of the iS3CC chip shown at the bottom, demonstrating daily progress of MARC culture during different phases of cerebral tissue formation. White arrows indicate connective neurite outgrowths, whereas white arrowheads indicate merged neurite bundles between spheroids. Dashed lines indicate the porous membrane separating the chambers. (d) Fluorescence pictures of intracellular calcium detected by fluo-4 direct, in cerebral tissues at day 25 and 42 and extended neurite outgrowths and bundles from both separated cultures, across the porous membrane indicated by horizontal dashed lines. (e) Live calcium imaging in both interconnected cerebral tissues in the chambers of the iS3CC chip, demonstrating active connections between the separated cerebral tissues. The kymographs of three ROIs (shown in corresponding color code in d, right) show neural activity of connections across the membrane as a function of time. Black arrows indicate the calcium transients during the imaging time. Scale bar: 60 s.

FIG. 4. Discharge propagation between MARC-produced cerebral tissues. (a,b) Two cerebral tissues were separately formed in the two chambers of an iS3CC chip, separated by a membrane (black dashed line). One of the chambers (left, “treated”) was treated with Penicillin G (“Pen”), whereas the other (right, “untreated”) was not. The activity of 522 neurons were detected in the treated (circles, left) and untreated (circles, right) tissues and analyzed by live calcium imaging (see also Supplementary Movie 3). Scale bar: 250 μm. (c,d) Time traces of normalized intensity (ΔF/F) from 20 representative neurons in the treated (c) and untreated (d) are shown. Time 0 refers to the addition of Penicillin G (“Pen”). The inset show zoom-in views of the pre-treatment time traces. Data from the 20 cells were offset for clarity. Crosses indicate detected transient spikes. Vertical scale bars: 1 ΔF/F. Horizontal scale bars: 60 s. (e,f) Time traces of 4 cells in the treated (lower) and untreated (upper) cerebral tissues pre- (e) and post-treatment (f) with Penicillin G. The black vertical lines indicate instances where all 4 cells in the treated tissue showed synchronized transient peaks. This synchronicity propagated ˜45% of the time to the cells in the untreated tissue. (g,h) Quantification of the change in fluorescence intensity (g) and fold change in neuronal activity (h, log scale) induced by addition of Penicillin G in the treated (left) and untreated (right) cerebral tissues. The symbols represent data for each cell, the boxes represent the median, 1^{st} and 3^{rd} quartiles, and the whiskers represent the 5^{th} and 95^{th} percentiles of the population data. Asterisk denotes statistically significant difference (Mann-Whitney U test, p<10⁻¹¹).

FIG. 5. Immunohistochemical co-staining of multiple markers on 100 μm sections of MARC-produced cerebral tissues at Day 90 revealed the overall distribution and regionalization of distinct neuronal cell types. Co-staining of neural progenitor cells (NPCs; PAX6 in red), early neurons (Tuj1 in green) and mature neurons (MAP2 in blue) (in greyscale, red is shown as grey, green as light grey and blue as dark grey) (a), co-staining of dopamine transporter (DAT in red) vesicular transporters of glutamine (VGLUT in green) and GABA (VGAT in blue) (b) co-staining GFAP (red), Olig2 (green) and MAP2 (blue) (c) and co-staining of dopamine transporter (DAT in red) and vesicular glutamine transporter (VGLUT in green) in particular (d i and ii) indicate the localized co-expression of these markers suggesting regionalization of these cell types in the cerebral tissues. Scale bars: 1 mm.

FIG. 6. Immediate fluorescence increase in the treated chamber upon Penicillin G treatment. Two cerebral tissues were separately formed via MARC protocol in the two chambers of an iS3CC chip, separated by a membrane (black dashed line). One of the chambers (left, “treated”) was treated with Penicillin G (“Pen”), whereas the other (right, “untreated”) was not treated. Scale bar: 1 mm.

FIG. 7. Measurement of particle transfer between the chambers of the iS3CC chip across the porous membrane. Fluorescein sodium salt with comparable molecular weight (376.27 g/mol) to Penicillin G sodium salt (367.37 g/mol), was added to one of the chambers of the iS3CC with the exact final concentration as Penicillin treatment (100 mg/ml) and the fluorescence of water in the other chamber was measured overtime using a plate reader (see Methods).

### DETAILED DESCRIPTION

### Definitions

A portion of this disclosure contains material that is subject to copyright protection (such as, but not limited to, diagrams, device photographs, or any other aspects of this submission for which copyright protection is or may be available in any jurisdiction.). The copyright owner has no objection to the facsimile reproduction by anyone of the patent document or patent disclosure, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

For purposes of the present invention, the following terms are defined below.

As used herein, the singular form terms “a,” “an,” and “the” include plural referents unless the content clearly dictates otherwise. Thus, for example, reference to “a cell” includes a combination of two or more cells, and the like. For example, a method for administrating a compound includes the administrating of a plurality of molecules (e.g. 10's, 100's, 1000's, 10's of thousands, 100's of thousands, millions, or more molecules).

As used herein, “about” and “approximately”, when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed invention.

As used herein, “and/or” refers to a situation wherein one or more of the stated cases may occur, alone or in combination with at least one of the stated cases, up to with all of the stated cases.

As used herein, “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc. As used herein, the term “at most” a particular value means that particular value or less. For example, “at most 5” is understood to be the same as “5 or less” i.e., 5, 4, 3, . . . −10, −11, etc.

As used herein, “comprising” or “to comprise” is construed as being inclusive and open ended, and not exclusive. Specifically, the term and variations thereof mean the specified features, steps or components are included. These terms are not to be interpreted to exclude the presence of other features, steps or components. It also encompasses the more limiting “to consist of”.

As used herein, “conventional techniques” or “methods known to the skilled person” refer to a situation wherein the methods of carrying out the conventional techniques used in methods of the invention will be evident to the skilled worker. The practice of conventional techniques in molecular biology, biochemistry, cell culture, genomics, sequencing, medical treatment, pharmacology, immunology and related fields are well-known to those of skill in the art. and are discussed, in various handbooks and literature references.

As used herein, “exemplary” means “serving as an example, instance, or illustration,” and should not be construed as excluding other configurations disclosed herein.

As used herein, “in vivo” refers to an event that takes place in a subject's body; “in vitro” refers to an event that takes places outside of a subject's body. For example, an in vitro assay or method encompasses any assay or method conducted outside of a subject. In vitro assays or methods encompass cell-based assays in which cells, alive or dead, are employed. In vitro assays also encompass a cell-free assay in which no intact cells are employed.

As used herein, “neural induction medium” refers to a medium causing the pluripotent stem cells to induce neuroectodermal differentiation. As used herein, “neural differentiation medium” refers to a medium further directing differentiation of the cells towards (mature-like) neural cells or tissue.

As used herein, “cell culture substrate” refers to a semi-solid, preferably gelatinous material or matrix, preferably comprising extracellular matrix components, e.g. basement membrane matrix components. Within the context of the current invention the cell culture substrate allows the cells to remain dispersed within the three dimensional form of the cell culture substrate after resuspension of the cells therein. The cell culture substrate thus allows cells dispersed therein to grow and differentiate.

The matrix provided by the cell culture substrate may thus refer to a three-dimensional network of extracellular macromolecules (natural or synthetic), such as collagen, enzymes, and glycoproteins, that provide structural and biochemical support of surrounding cells (in vitro). Non-limiting examples of such cell culture substrates included for instance commercially available gels or hydrogels such as Matrigel rgf, BME1, BME1rgf, BME2, BME2rgf, BME3 (all Matrigel variants) Collagen I, Collagen IV, mixtures of Collagen I and IV, or mixtures of Collagen I and IV, and Collagen II and III), puramatrix, hydrogels, Cell-Tak™, Collagen I, Collagen IV, Matrigel® Matrix, Fibronectin, Gelatin, Laminin, Osteopontin, Poly-Lysine (PDL, PLL), PDL/LM and PLO/LM, PuraMatrix® or Vitronectin. In one preferred embodiment, the matrix components are obtained as the commercially available Corning® MATRIGEL® Matrix (Corning, NY 14831, USA). The term “MATRIGEL® Matrix” as used herein refers to a non-limiting example of a matrix that is extracted from the Engelbreth-Holm-Swarm (“EHS”) mouse tumor, a tumor rich in basement membrane. The major matrix components are laminin, collagen IV, entactin, and heparin sulfate proteoglycan (“HSPG”). The matrix also contains growth factors, matrix metalloproteinases (collegenases), and other proteinases (plasminogen activators), as well as some as yet undefined extracellular matrix components. At room temperature, MATRIGEL® Matrix gels to form a reconstituted basement membrane.

As used herein, “pluripotent stem cell” refers to a stem cell capable of producing all cell types of the organism and can produce cells of the germ layers, e.g. endoderm, mesoderm, and ectoderm, of a mammal and encompasses at least pluripotent embryonic stem cells and induced pluripotent stem cells. Pluripotent stem cells can be obtained in different ways. Pluripotent embryonic stem cells may, for example, be obtained from the inner cell mass of an embryo. Induced pluripotent stem cells (iPSCs) may be derived, for example by chemical reprogramming, from somatic cells. (Pluripotent) stem cells may also be in the form of an established cell line or be non-embryonic (adult) stem cells.

As used herein, the term “three dimensional culture” or 3D culture refers to a method of culturing cells or tissues wherein cells or tissues are implanted (resuspended, dispersed, embedded) into an artificial structure (here referred to as cell culture substrate) capable of supporting three-dimensional tissue formation.

As used herein, “Small Mothers Against Decapentaplegic” or “SMAD” refers to a signalling molecule. As used herein a SMAD protein signalling inhibitor is a compound that downregulates expression or activity of SMAD.

As used herein, an activator of Wnt signalling is a compound that upregulates expression or activity of Wnt.

As used herein a GSK-3 inhibitor is a compound that downregulates expression or activity of GSK-3.

As used herein, an activator of SHH signalling is a compound that upregulates expression or activity of Sonic Hedgehog.

It is contemplated that any method, use or composition described herein can be implemented with respect to any other method, use or composition described herein. Embodiments discussed in the context of methods, use and/or compositions of the invention may be employed with respect to any other method, use or composition described herein. Thus, an embodiment pertaining to one method, use or composition may be applied to other methods, uses and compositions of the invention as well.

As embodied and broadly described herein, the present invention is directed to the surprising finding of a method for obtaining (three dimensional) (neural) tissue composition having highly advantageous and desirable properties.

The method is as defined and described in the claims, taking into account the definitions as provided herein. Advantages of the method as well as particular details of the method are provided in the Examples below.

The skilled person is capable in determining further suitable conditions for performing the invention, for example with respect to concentrations of compounds, suitable culture mediums, suitable temperature and devices to use in the context of the current invention.

It will be understood that the information in the Examples is not to be construed as to be limiting any of the claims as provided herein or as to indicate essential technical features beyond the features provided in the claims.

It will be understood that all details, embodiments and preferences discussed with respect to one aspect or embodiment of the invention is likewise applicable to any other aspect or embodiment of the invention and that there is therefore not need to detail all such details, embodiments and preferences for all aspect separately.

Having generally described the invention in the claims, the same will be more readily understood through reference to the following examples which is provided by way of illustration and is not intended to be limiting of the present invention.

Various features of a developing central nervous system, including sensory input-output, neuronal migration and regionalization have been recapitulated through recent advances in the culture of brain organoids. These organoids can model the fundamental processes in brain development and disease. Generation of current organoid models originate from classic dissociation-reaggregation paradigms, often relying on mechanically-enforced quick reaggregation of pluripotent stem cells. in vivo.

However, a remaining challenge is to obtain in vitro complex neuronal networks with functional interconnectivity such as found in vivo, in native brain tissue. The current inventors realized that an important step in the assembly of neuronal circuits and mature interconnected networks is neuronal migration. The current inventors now aimed to develop multi-regional brain tissues in vitro, mimicking complex neuronal networks with functional interconnectivity such as found in vivo. The means and methods as provided by the invention now allow to provide for complex neuronal networks such as found in vivo. The three dimensional neural tissue as provided by the invention was shown to have characteristics of mature neuronal networks, including synchronized influxes of extracellular calcium and modular functional connectivity patterns, demonstrating the formation of interconnected network.

Hence, the in vitro methods of the invention are to provide for three dimensional neural tissue. Such three dimensional neural tissue preferably is a mature neuronal network. Such neural tissue or mature neuronal networks are highly preferably human tissue. More preferably, such neural tissue or mature neuronal networks are at the mesoscale. With mesoscale, a size of the generated tissue in the order of magnitude of millimeters in size is indicated, e.g. in the range of 2-4 mm. As shown in the example section, such generated neural tissue was found to be highly useful for studying neurological disorders, in particular of human neurological disorders, such as epilepsy.

Protocols available for the culturing organoids inherently suppress such processes and/or do not allow to provide for interconnected networks. Conventional methods use a quick and mechanically enforced aggregation of dissociated cells. In contrast thereto, the methods of the invention involve the formation of three dimensional neural tissue by active (migrative) reaggregation of cells during induced differentiation with the support of a cell culture substrate, e.g. a matrix. The process may also be referred to as so called matrix-supported active (migrative) reaggregation of cells (MARC). Hence, cells are first subjected to neural induction in two dimensions, and subsequently dissociated and subjected to neural differentiation with the support of a cell culture substrate in three dimensions.

Hence, in one embodiment, the invention provides for an in vitro method of producing a three dimensional neural tissue composition, the method comprising the steps of
re-suspending pluripotent stem cells or cells that are obtained by culturing pluripotent stem cells in a neural induction medium in a cell culture substrate, preferably wherein the re-suspended cells are dispersed in the cell culture substrate, and
inducing re-aggregation and/or differentiation of the cells that are resuspended in the cell culture substrate, preferably by culturing the cells that are resuspended in the cell culture substrate in the presence of a neural differentiation medium.

The pluripotent stem cells or cells are first re-suspended, i.e. the cells or pluripotent stem cells that have been cultured in a neural induction medium. Re-suspension is understood to involve the detachment of the cells that have been cultured in neural induction medium cells. Such detachment can be carried out by methods well know in the art, e.g. physical resuspension and/or by using solutions comprising EDTA and proteases such as trypsin or the like. This way, the cells can be conveniently collected and dispersed in a cell culture substrate.

Within the cell culture substrate, subsequently re-aggregation and/or differentiation is induced. Preferably this is in the presence of a neural differentiation medium.

It is understood that a neural induction medium refers to a medium causing the pluripotent stem cells to indicate neuroectodermal differentiation. A neural differentiation medium refers to a medium further directing differentiation of the cells towards (mature-like) neural cells or tissue. These cell culture media differ in composition. The protocol as devised by the current inventors involves a phased introduction and withdrawal of culture components. In this way, the highly advantageous three dimensional neural tissue composition can be obtained.

Pluripotent stem cells may optionally be used directly and re-suspended in the cell culture substrate, or pluripotent stem cells are first cultured in a neural induction medium, prior to re-suspending the cells. When pluripotent stem cells are used directly, the cells resuspended in the cell culture substrate are subjected to at least one neural induction medium and followed by at least one neural differentiation medium. After the culturing in neural differentiation medium, cells can be cultured in neural maintenance medium.

Hence, in another embodiment, in accordance with the invention an in vitro method is provided of producing a three dimensional neural tissue composition, the method comprising the steps of
a) providing pluripotent stem cells;
b) optionally, culturing the pluripotent stem cells in the presence of at least one neural induction medium;
c) re-suspending the cells of step a) or b) in a cell culture substrate, preferably wherein the re-suspended cells are dispersed in the cell culture substrate;
d) inducing re-aggregation and/or differentiation of the cells that are resuspended in the cell culture substrate, preferably by culturing the cells that are resuspended in the cell culture substrate in the presence of at least one neural differentiation medium or in the presence of at least one neural induction medium followed by culturing in the presence of at least one neural differentiation medium;
e) optionally, culturing the cells of step d) in the presence of at least one neural maintenance medium.

The pluripotent stem cells preferably are human pluripotent stem cells. It is understood that the means and methods of the invention allows for utilizing human pluripotent stem cells, human induced pluripotent stem cells, or human embryonic stem cells. The means and methods also allow for using non-human pluripotent stem cells, non-human induced pluripotent stem cells, non-human embryonic stem cells, or non-human non-embryonic stem cells.

Hence, in a further embodiment, in an in vitro method according to the invention, the pluripotent stem cells are human pluripotent stem cells, non-human pluripotent stem cells, human induced pluripotent stem cells, non-human induced pluripotent stem cells, human embryonic stem cells, non-human embryonic stem cells, human non-embryonic stem cells, or non-human non-embryonic stem cells.

In another further embodiment, in an in vitro method in accordance with the invention, the pluripotent stem cells are cultured until at least 60-80% confluence, in the presence of a pluripotent stem cell proliferation medium before culturing the cells in the presence of the at least one neural induction medium. With regard to confluence it is understood that this is a measure of the density of cells attached to the surface on which the cells grow. A confluence of 20% means that 20% of the surface on which the cells grow is covered with cells. It may be preferred to have the pluripotent cells divide and expand to a confluence of at least 60% prior to culturing the cells in the presence of the at least one neural induction medium. Culturing of the pluripotent cells to the confluence of at least 60% can be performed such as described in the examples, and can be under feeder-free conditions.

In any case, once suitable pluripotent stem cells, which preferably are human pluripotent stem cells such as human induced pluripotent stem cells are provided, the cells can be cultured in a neural induction medium. As said, a neural induction medium refers to a medium causing the pluripotent stem cells to induce neuroectodermal differentiation. Preferably, such a medium comprises one or more of a compound selected from the group of a compound that inhibits Small Mothers Against Decapentaplegic (SMAD) protein signaling (“SMAD inhibitor”), a compound that activates Wnt-signaling, a compound that activates Sonic Hedgehog signaling (“SHH activator”), and a basic Fibroblast Growth Factor (“bFGF”). More preferably, such a medium comprises a compound that inhibits Small Mothers Against Decapentaplegic (SMAD) protein signaling (“SMAD inhibitor”), a compound that activates Wnt-signaling, a compound that activates Sonic Hedgehog signaling (“SHH activator”), and a basic Fibroblast Growth Factor (“bFGF”). The current invention also provides for a neural induction medium as defined herein.

Hence, in a further embodiment, in an in vitro method in accordance with the invention, the neural induction medium comprises:
a) at least one compound that inhibits Small Mothers Against Decapentaplegic (SMAD) protein signaling (“SMAD inhibitor”), preferably wherein said at least one SMAD inhibitor is selected from the group consisting of dorsomorphin, SB431542, noggin, LDB193189, or any combination thereof, even more preferably wherein said at least one SMAD inhibitor comprises dorsomorphin and SB431542;
b) at least one compound that activates Wnt-signaling, preferably wherein said compound inhibits Glycogen synthase kinase 3 (“GSK-3 inhibitor”), preferably wherein said GSK-3 inhibitor is selected from the group consisting of CHIR99021, CHIR98014, and 6-bromoindirubin-3′-oxime;
c) at least one compound that activates Sonic Hedgehog signaling (“SHH activator”), preferably wherein said SSH activator is selected from the group consisting of a SSH protein, pumorphamine, SAG smoothened agonist, and Hh-Ag1.5; and/or;
d) basic Fibroblast Growth Factor (“bFGF”).

In yet another embodiment, in an in vitro method in accordance with the invention, the neural induction medium comprises:
e) at least one compound that inhibits Small Mothers Against Decapentaplegic (SMAD) protein signaling (“SMAD inhibitor”), preferably wherein said at least one SMAD inhibitor is selected from the group consisting of dorsomorphin, SB431542, noggin, LDB193189, or any combination thereof, even more preferably wherein said at least one SMAD inhibitor comprises dorsomorphin and SB431542;
f) at least one compound that activates Wnt-signaling, preferably wherein said compound inhibits Glycogen synthase kinase 3 (“GSK-3 inhibitor”), preferably wherein said GSK-3 inhibitor is selected from the group consisting of CHIR99021, CHIR98014, and 6-bromoindirubin-3′-oxime;
g) at least one compound that activates Sonic Hedgehog signaling (“SHH activator”), preferably wherein said SSH activator is selected from the group consisting of a SSH protein, pumorphamine, SAG smoothened agonist, and Hh-Ag1.5; and;
h) basic Fibroblast Growth Factor (“bFGF”).

In still another embodiment, the neural induction medium comprises dorsomorphin and SB431542, CHIR99021, Hh-Ag1.5, and bFGF”.

In yet another embodiment, the invention provides for an in vitro method wherein the at least one neural induction medium comprises at least one SMAD inhibitor, at least one GSK-3 inhibitor, at least one SHH activator, and bFGF, preferably wherein the neural induction medium comprises dorsomorphin, SB431542, CHIR99021, SHH, and b-FGF. In a further embodiment, neural induction medium is provided comprising, SB431542 (Tocris, 1614), Dorsomorphin dihydrochloride (Tocris, 3093), CHIR99021 (Sigma, SML1046), mouse recombinant Sonic Hedgehog (SHH)-C2511 (Genscript, Z03050-50), and basic fibroblast growth factor (b-FGF). Still further, neural induction medium is provided comprising, 10 μM SB431542 (Tocris, 1614), 1 μM Dorsomorphin dihydrochloride (Tocris, 3093), 10 μM CHIR99021 (Sigma, SML1046), 100 ng/ml mouse recombinant Sonic Hedgehog (SHH)-C2511 (Genscript, Z03050-50), and 10 ng/ml basic fibroblast growth factor (b-FGF). In yet another embodiment, neural induction medium is provided as defined in the example section.

The pluripotent stem cells are cultured in the presence of the at least one neural induction medium. It is understood that “at least one” includes using one neural induction medium defined with regard to the at least one SMAD inhibitor, at least one GSK-3 inhibitor, at least one SHH activator, and bFGF, having the same composition. The neural induction medium, like any medium as defined herein, can be refreshed during the culture process, e.g. by removing the neural induction medium and optionally washing the cells e.g. with PBS, and adding fresh neural induction medium to the cells. It is understood that with “at least one” multiple neural induction media with varied compositions may be contemplated.

In further embodiments, in the in vitro method in accordance with the invention, culturing the pluripotent stem cells in the presence of at least one neural induction medium is for a period of at least 2, 3, 4, or 5 days, preferably between 2-15 days, 3-10 days or 4-9 days. In another embodiment, in the in vitro method in accordance with the invention, culturing the pluripotent stem cells in the presence of at least one neural induction medium is for a period of at least 2, 3, 4, or 5 days. In yet another embodiment, in the in vitro method in accordance with the invention, culturing the pluripotent stem cells in the presence of at least one neural induction medium is for a period of at least 2 days, at least 3 days, at least 4 days or at least 5 days. In one embodiment, the period is 2 days. In one embodiment, in the in vitro method in accordance with the invention, culturing the pluripotent stem cells in the presence of at least one neural induction medium is for a period of between 2-15 days. In one embodiment, in the in vitro method in accordance with the invention, culturing the pluripotent stem cells in the presence of at least one neural induction medium is for a period of between 3-10 days. In another embodiment, in the in vitro method in accordance with the invention, culturing the pluripotent stem cells in the presence of at least one neural induction medium is for a period of between 4-9 days.

In any case, an appropriate induction period is selected to allow for the (human) pluripotent stem cells to initiate neural differentiation. The pluripotent stem cells are cultured on an appropriate substrate and cells are preferably grown in two dimensions. Hence, pluripotent stem cells cultured to a confluence of at least 60%, can be maintained on the same substrate, i.e. do not require dislodging of the cells and seeding the cells to e.g. a new culture dish. As cells can grow to high density, refreshing culture medium daily, such as described in the example section can be contemplated. Hence, in another embodiment, in the in vitro method in accordance with the invention, culturing the pluripotent stem cells in the presence of at least one neural induction medium is performed in two dimensions, so called “2D culturing”.

After culturing the cells in neural induction medium, the cells are cultured in three dimensions. Cells are re-suspended in a cell culture substrate. In this next step, the cells are cultured in three dimensions, so called “3D culturing”, to obtain the three dimensional tissue composition in accordance with the invention. The cell culture substrate provides for a suitable environment that allows for culturing in three dimensions. In a further embodiment, suitable cell culture substrates that can be contemplated in accordance with the invention comprises extracellular matrix components and/or wherein the cell culture substrate comprises Matrigel, gelatin, vitronectin, laminin, fibronectin, and/or collagen, preferably the cell culture substrate is Matrigel. In one embodiment, the cell culture substrate comprises extracellular matrix components. In another embodiment, the cell culture substrate comprises extracellular matrix components, gelatin, vitronectin, laminin, fibronectin, and/or collagen. In yet another embodiment, the cell culture substrate comprises extracellular matrix components, gelatin, vitronectin, laminin, fibronectin, and collagen. As said, it may be preferred to have Matrigel (Corning 734-0269), or the like, as a cell culture substrate.

Hence, in a further embodiment, the invention provides for an in vitro method wherein the pluripotent stem cells, or the cells obtained after culturing of induced pluripotent stem cells in the neural induction medium, are obtained, preferably by preparing a cell suspension, and resuspended in the cell culture substrate, preferably wherein the re-suspended cells are dispersed in the cell culture substrate, preferably wherein the cell culture substrate comprises extracellular matrix components and/or wherein the cell culture substrate comprises Matrigel, gelatin, vitronectin, laminin, fibronectin, and/or collagen, preferably the cell culture substrate is Matrigel.

As said, the pluripotent stem cells, or the cells, preferably dispersed in the cell culture substrate as defined above, are subsequently subjected to at least one neural differentiation medium. The current invention, as said, provides for an intricate neuronal differentiation protocol which employs phased introduction and withdrawal of culture additives. Hence, in this embodiment, the at least one neural differentiation medium comprises culturing the cells in a first and a subsequent second neural differentiation medium, said first and second neural differentiation having a different composition. The first neural differentiation medium preferably comprises b-FGF, at least one SHH activator, and a Fibroblast growth factor 8 protein (“FGF8”). More preferably, the first neural differentiation medium comprises b-FGF, SSH protein, and a Fibroblast growth factor 8 protein (“FGF8”). The second neural differentiation medium preferably comprises at least one SHH activator, preferably SSH protein, and a Fibroblast growth factor 8 protein (“FGF8”), and is substantially free of b-FGF. In another embodiment, the second neural differentiation medium comprises SSH protein, and a Fibroblast growth factor 8 protein (“FGF8”), and is substantially free of b-FGF. In yet another embodiment, the second neural differentiation medium comprises SSH protein and a Fibroblast growth factor 8 protein (“FGF8”). Hence, in another embodiment, a first neural differentiation medium is provided comprising b-FGF, at least one SHH activator, and a Fibroblast growth factor 8 protein (“FGF8”) and a second neural differentiation medium is provided comprising SSH protein, and a Fibroblast growth factor 8 protein (“FGF8”). In a further embodiment, a first neural differentiation medium is provided comprising, b-FGF, SHH-C2511, and human recombinant FGF8 (Gibco, PHG0184) and a second neural differentiation medium comprising SHH-C2511 and human recombinant FGF8 (Gibco, PHG0184). In yet another a further embodiment, a first neural differentiation medium is provided comprising, 10 ng/ml b-FGF, 20 ng/ml SHH-C2511, and 100 ng/ml human recombinant FGF8 (Gibco, PHG0184) and a second neural differentiation medium comprising 20 ng/ml SHH-C2511 and 100 ng/ml human recombinant FGF8 (Gibco, PHG0184). In still another embodiment, a first and a second neural differentiation medium is provided as defined in the example section.

Accordingly, in a further embodiment, an in vitro method in accordance with the invention is provided wherein culturing the resuspended cells in the presence of at least one neural differentiation medium in step d) comprises:
i) culturing the resuspended cells in the presence of a first neural differentiation medium;
ii) culturing the resuspended cells in the presence of a second neural differentiation medium;
   wherein said first, and second neural differentiation medium each have a different composition, preferably wherein:
the first neural differentiation medium comprises b-FGF, at least one SHH activator, preferably SSH protein, and a Fibroblast growth factor 8 protein (“FGF8”); and
the second neural differentiation medium comprises at least one SHH activator, preferably SSH protein, and a Fibroblast growth factor 8 protein (“FGF8”), and is substantially free of b-FGF.

The step of culturing the resuspended cells in the presence of at least one neural differentiation medium is for a period of at least 5, 6, or 7 days. Preferably, for a period of between 5-45 days, more preferably 8-35 days or most preferably 10-25 days. As said, the at least one neural differentiation medium comprises preferably culturing in a first and subsequent second neural differentiation medium as defined above. Accordingly, in a further embodiment, in the in vitro method in accordance with the invention, culturing the resuspended cells in the presence of the first neural differentiation medium is for a period that is shorter than the period for culturing in the presence of the second neural differentiation medium, preferably wherein culturing in the presence of the first neural differentiation medium is for a period between 1 and 10 days and/or wherein culturing in the presence of the second neural differentiation medium is for a period of between 5 and 30 days. Preferably, the cells are cultured in the first neural differentiation medium for about a week, and in the second for about two weeks. After culturing the cells in the at least one neural differentiation medium, the three dimensional neural tissue composition is formed. As shown in the example section, such three dimensional neural tissue composition can be large cerebral tissue with a size in the millimeter scale, for example having a cross section of 2-4 millimeter.

Subsequently, the cells can be cultured in at least one neural maintenance medium. Neural maintenance medium may have the same composition as neural differentiation medium, but being substantially free of SHH activator, a Fibroblast growth factor 8 protein (“FGF8”) and b-FGF. Hence, in a further embodiment, the at least one neural maintenance medium is substantially free of an SHH activator, preferably SSH protein, a Fibroblast growth factor 8 protein (“FGF8”) and b-FGF. Hence, in one embodiment, neural maintenance medium is provided which is substantially free of an SHH activator, preferably SSH protein, a Fibroblast growth factor 8 protein (“FGF8”) and b-FGF. As the name implies when cultured in neural maintenance medium, the three dimensional neural tissue that was formed is maintained and continues to grow. The tissue can be maintained for long period in vitro. In a further embodiment, the cells are cultured in the presence of at least one neural maintenance medium for a period of at least 10, 15, 25, 40, 80 or 90 days.

It is understood that in accordance with the invention, the three dimensional neural tissue that can be obtained with the methods of the invention allows for the study of neurological disorders. For example, in case such a disorder is a genetic disorder, such tissue can be easily be prepared in vitro by carrying out the methods of the invention and utilizing pluripotent stem cells of an appropriate (human) donor having the genetic disorder, or genetically engineered cells (e.g. provide pluripotent stem cells of a healthy donor and genetically modify these). Once prepared, interventions can be tested in the in vitro setting in order to test their effect. Interventions may also be tested during the preparations of such tissue. Alternatively, means and methods may be applied to trigger phenotypes of neurological disorders in vitro. As shown in the example section, three dimensional neural tissue as provided in accordance with the invention, when subjected to Penicillin G, results in the formation of neural tissue which recapitulates in vitro abnormal signal transmission, like observed in epilepsy (epileptiform discharge propagation). Hence, three dimensional neural tissue as provided in accordance with the invention, when subjected to Penicillin G, can provide for a three dimensional neural tissue which is highly useful for studying of epilepsy.

Hence, in a further embodiment, in the methods in accordance with the invention, neural three dimensional tissue is provided for studying epilepsy by treating the three dimensional neural tissue with Penicillin G.

In a further embodiment, three dimensional neural tissue is provided obtainable by any of the methods as described herein. Such three dimensional neural tissue derived from pluripotent stem cells is prepared in vitro and has highly advantageous properties.

Hence, in another embodiment, the invention provides for three dimensional neural tissue compositions, prepared in vitro from pluripotent stem cells, wherein the tissue expresses markers of neural progenitor cells, early and mature neurons, mature GABAergic neurons, mature dopaminergic neurons, astrocytes and oligodendrocytes. In a preferred embodiment, the invention provides for three dimensional neural tissue compositions, prepared in vitro from pluripotent human stem cells, wherein the tissue expresses markers of neural progenitor cells, early and mature neurons, mature GABAergic neurons, mature dopaminergic neurons, astrocytes and oligodendrocytes.

The invention provides for three dimensional neural tissue compositions, prepared in vitro from pluripotent stem cells, wherein the tissue is multiregional cerebral tissue, expressing markers of neural progenitor cells, early and mature neurons, mature GABAergic neurons, mature dopaminergic neurons, astrocytes and oligodendrocytes.

Furthermore, wherein said multiregional cerebral tissue comprises interconnective neurons. Such interconnective neurons forming a functional neuronal network. Interconnective neurons are characterized in showing synchronized neuronal firing, e.g. as shown herein in the examples.

Said three dimensional neural tissue in accordance with the invention having neuronal interconnectivity is useful for studying signal transmission between interconnect neural tissues. Such interconnected neural tissues may be provided by culturing in each of two separate chambers, separated by a porous membrane. Hence, the cell culture substrate as defined herein is comprised in the two separate chambers, and the steps of the methods carried out as defined herein. By having a membrane between the two chambers, with a pore size that allows neurite interconnections forming between the two chambers, the separated tissues can connect. For example, a pore size of about 8 μm can allow for neurite interconnections between neural tissues. This way, signal transmission between interconnected neural tissues can be studied.

As shown in the examples, such interconnected cerebral tissues are highly useful in studying neurological disorders. Hence, in another embodiment, the methods of the invention provide for at least two interconnected neural tissues, wherein the at least two interconnected tissues are prepared by providing cell culture substrates in at least two separate chambers comprising the cell culture substrate, the two separate chambers being separated by a membrane that allows neurite interconnection formation.

Also provided are media suitable for carrying the methods in accordance with the invention. Hence, in another embodiment, the invention provides for:
neural induction medium;
a first neural differentiation medium;
a second neural differentiation medium; and
a neural maintenance medium,
   wherein the neural induction medium comprises
a) at least one compound that inhibits Small Mothers Against Decapentaplegic (SMAD) protein signaling (“SMAD inhibitor”), preferably wherein said at least one SMAD inhibitor is selected from the group consisting of dorsomorphin, SB431542, noggin, LDB193189, or any combination thereof, even more preferably wherein said at least one SMAD inhibitor comprises dorsomorphin and SB431542;
b) at least one compound that activates Wnt-signaling, preferably wherein said compound inhibits Glycogen synthase kinase 3 (“GSK-3 inhibitor”), preferably wherein said GSK-3 inhibitor is selected from the group consisting of CHIR99021, CHIR98014, and 6-bromoindirubin-3′-oxime;
c) at least one compound that activates Sonic Hedgehog signaling (“SHH activator”), preferably wherein said SSH activator is selected from the group consisting of a SSH protein, pumorphamine, SAG smoothened agonist, and Hh-Ag1.5; and;
d) basic Fibroblast Growth Factor (“b-FGF”);
   wherein the first neural differentiation medium comprises:
b-FGF, at least one SHH activator, preferably SSH protein, and a Fibroblast growth factor 8 protein (“FGF8”);
   wherein the second neural differentiation medium comprises:
at least one SHH activator, preferably SSH protein, and a Fibroblast growth factor 8 protein (“FGF8”), and is substantially free of b-FGF; and
   wherein the neural maintenance medium is substantially free of SHH activator, a Fibroblast growth factor 8 protein (“FGF8”) and b-FGF.

As said, also provided are media suitable for carrying the methods in accordance with the invention. In another embodiment, the invention provides for one or more of a:
neural induction medium;
a first neural differentiation medium;
a second neural differentiation medium; and
a neural maintenance medium.

Herein, the neural induction medium comprises
e) at least one compound that inhibits Small Mothers Against Decapentaplegic (SMAD) protein signaling (“SMAD inhibitor”), preferably wherein said at least one SMAD inhibitor is selected from the group consisting of dorsomorphin, SB431542, noggin, LDB193189, or any combination thereof, even more preferably wherein said at least one SMAD inhibitor comprises dorsomorphin and SB431542;
f) at least one compound that activates Wnt-signaling, preferably wherein said compound inhibits Glycogen synthase kinase 3 (“GSK-3 inhibitor”), preferably wherein said GSK-3 inhibitor is selected from the group consisting of CHIR99021, CHIR98014, and 6-bromoindirubin-3′-oxime;
g) at least one compound that activates Sonic Hedgehog signaling (“SHH activator”), preferably wherein said SSH activator is selected from the group consisting of a SSH protein, pumorphamine, SAG smoothened agonist, and Hh-Ag1.5; and;
h) basic Fibroblast Growth Factor (“bFGF”);
   wherein the first neural differentiation medium comprises:
b-FGF, at least one SHH activator, preferably SSH protein, and a Fibroblast growth factor 8 protein (“FGF8”);
   wherein the second neural differentiation medium comprises:
at least one SHH activator, preferably SSH protein, and a Fibroblast growth factor 8 protein (“FGF8”), and is substantially free of b-FGF; and
   wherein the neural maintenance medium is substantially free of SHH activator, a Fibroblast growth factor 8 protein (“FGF8”) and b-FGF.

Accordingly, the invention also provides for a neural induction medium as defined herein, or a first or second neural differentiation medium as defined herein. These neural induction media as defined herein are in particular useful for use in the methods as defined herein, i.e. for use in preparing three dimensional neural tissue in vitro.

In any case, the three dimensional neural tissue, cerebral tissue, multiregional cerebral tissue, interconnected tissue, or cerebral tissue as prepared and provided in accordance with the invention is highly useful in studying neurological disorders. Hence, in another embodiment, the use is provided of three dimensional neural tissue, cerebral tissue, multiregional cerebral tissue, interconnected tissue, or cerebral tissue, as prepared and provided in accordance with the invention, for studying neurological disorders, healthy tissue studies and/or neuronal network studies. In another embodiment, the invention provides for the use of the three dimensional neural tissue composition or cerebral tissue as provided and prepared in accordance with the invention, in neurological disorder studies, in healthy tissue studies and/or in neuronal network studies.

As said one of the key elements of the development of nervous systems is the formation of complex neuronal network. The three dimensional neural tissues as provided and prepared in accordance with the invention can show characteristics of mature neuronal networks, including synchronized influxes of extracellular calcium and modular functional connectivity patterns, demonstrating the formation of interconnected network within the intact tissues. As such, the three dimensional neural tissue in accordance with the invention can be used to study physiological and pathophysiological features of healthy and diseased neuronal networks. Examples of disorders that can studied include epilepsy, Alzheimer's disease, schizophrenia, multiple sclerosis, depression, ASD, and traumatic brain injury.

### Examples

Human cerebral tissues created via active cellular reaggregation produce functionally interconnected 3D neuronal network to mimic pathological circuit disturbance Various characteristics of a developing central nervous system, including sensory input-output^{1,2}, neuronal migration^{3,4}, and regionalization^{3,5}, have been recapitulated through recent advances in the culture of brain organoids. These organoids can model the fundamental processes in brain development and disease. A remaining critical challenge, however, is to achieve complex neuronal networks with functional interconnectivity as in native brain tissue. Generation of current organoid models originates from classic dissociation-reaggregation paradigms⁶, often relying on mechanically-enforced quick reaggregation of pluripotent stem cells⁷. Here we describe an alternative method that promotes matrix-supported active (migrative) reaggregation of cells (MARC), reminiscent of in vivo developmental morphing processes, to engineer multi-regional brain tissues in vitro. Measurements of neuronal activity in intact 3D tissues revealed functional interconnectivity, characteristic of cerebral neuronal networks. As a proof of concept, we here show that interconnected cerebral tissues produced using this approach can mimic propagation of epileptiform discharges in a custom-built in-vitro platform.

### Materials and Methods

### Cell Culture.

Human induced pluripotent stem cells (hiPSCs) were cultured in mTeSR medium (STEMCELL Technologies). The cell culture flasks were coated with Matrigel (hECS-qualified matrix, Corning, C354277) diluted in a 1:1 mixture of Dulbecco's Modified Eagle's Medium (DMEM, Gibco) and Ham's F-12 Nutrient Mixture (Gibco) with a v/v ratio of 1:80 for 2 hours in an incubator at 37° C. and 5% CO₂. On the first day of culture, the hiPSCs were treated with 10 μM ROCK inhibitor Y-27632 (STEMCELL Technologies). The cells were washed using Dulbecco's Phosphate-Buffered Saline (DPBS, Gibco) and the medium was refreshed daily until 80-100% confluence was reached.

### Neural Induction.

The cells were switched to neural induction medium containing 1:1 mixture of N2/B27 medium containing 10 ng/ml basic fibroblast growth factor (b-FGF), 1 μM Dorsomorphin dihydrochloride (Tocris, 3093), 10 μM SB431542 (Tocris, 1614), 100 ng/ml mouse recombinant Sonic Hedgehog (SHH)-C2511 (Genscript, Z03050-50), and 10 μM CHIR99021 (Sigma, SML1046). N2 medium consisted of DMEM/F12 medium (Gibco) with 1×N2 supplement (Gibco, 17502048), 5 μg/ml insulin (Sigma, 19278), 1 mM L-Glutamine (Lonza, 17605E), 100 μM MEM-Non-Essential Amino Acid solution (NEAA) (Gibco), 100 μM 2-mercaptoathanol (Sigma, M3148), and 1:100 Penicillin-Streptomycin (Lonza, 17602E). B27 medium consisted of Neurobasal medium (Gibco) and 1×B27 supplement (Gibco, 17504044). The cells were washed daily using DPBS and maintained in induction medium.

### Supported Reaggregation and Tissue Formation.

Neural differentiation. After 1 week of neural induction, the cells were dissociated using Accutase (STEMCELL Technologies, 07920) and resuspended in growth factor reduced (GFR) Matrigel (Corning, 734-0269) and neural differentiation medium with a 70:30 v/v ratio at a density of 50,000 cells/chamber. Neural differentiation medium consisted of N2/B27 medium containing 10 ng/ml b-FGF, 20 ng/ml SHH-C2511, and 100 ng/ml human recombinant FGF8 (Gibco, PHG0184). The medium was refreshed daily for 7-10 days as the cells aggregated to form spheroids.

Pre-terminal differentiation and neural maintenance. Following spheroid formation, the medium was replaced with pre-terminal differentiation medium containing neural differentiation medium without b-FGF. After 10-15 days of culture, during which period the spheroids extended neurites and made extensive connections with each other, the medium was replaced with N2/B27 medium, entering the terminal differentiation phase. From this point, the medium was changed every other day.

iS3CC Device Fabrication.

The three-dimensional model of the device was built in Siemens NX (version NX10) software, from which the model of the negative mold was created. A polycarbonate (PC) negative mold was fabricated using micro-milling (Mikron wf 21C). PDMS silicon elastomer kit (Sylgard 184) was used to create the devices using soft lithography. A solution of silicon elastomer and curing agent with a weight ratio of 10:1 was mixed and degassed and then poured into the molds and cured in the oven at 80° C. for 3 hours. After that, polyethylene terephthalate (PET) porous membranes with a pore size of 8 μm (ThinCert, 657638) were cut into desired size and placed in the right position in the chip. Gluing and immobilization of the chip and the membrane on a 0.17 mm glass slide was applied using PDMS mixture with the same composition as mentioned earlier. The chips had a final cure in the oven at 110° C. for 2 hours.

Cerebral Tissue Formation and Maintenance in the iS3CC Chips.

The transparent PDMS devices were repeatedly washed using 70% ethanol followed by sterilization using UV light in the safety cabinets (3×5 min). After neuronal induction phase, the cells were disassociated using Accutase and resuspended in 35 μl of a mixture of cold GFR-Matrigel and differentiation medium with a 70:30 v/v ratio at a final concentration of 50,000 cells per chamber. The chips containing cells and Matrigel-medium mixture were placed in a Petri dish to prevent contamination, since the chips have an open top, and kept in an incubator at 37° C. and 5% CO₂ for 5 min to polymerize the Matrigel mixture. After that, an additional 200 μl of differentiation medium was added to each chamber and the chips were placed back into the incubator. The medium was refreshed and the culture was continued as described above, with gentler handing to prevent damage to the gel and cells.

### Immunohistochemistry.

The cerebral tissues were fixed in 3.7% paraformaldehyde for 2 hours at 4° C. and washed five times with PBS for 10 min. After that they were gently detached and taken out of the chambers and placed in cryomolds (Tissue-Tek). After that, OCT compound (Tissue-Tek) embedding medium was added to the cerebral tissues and snap-frozen on dry ice. Sections of 10 μm and 100 μm were created using cryotome (Microm, HM 550). For immunostaining, the sections were dried at room temperature and subsequently permeabilized for 10 min using 0.5% Triton X-100 in PBS and blocked 2×10 min using 10% normal donkey serum. After that, sections were incubated in the following primary antibodies diluted in PBS containing 1% normal donkey serum: rabbit polyclonal anti-Pax6 (1:200, Invitrogen, 42-6600), mouse monoclonal anti-beta-Tubulin-Ill, Tuj1 (1:200, Merck, MAB1637), chicken polyclonal to tyrosine hydroxylase, TH (1:200, Abcam, ab76442), rabbit monoclonal to Glutaminase (1:200, Thermofisher, 701965), mouse monoclonal anti vesicular glutamate transporter-I, VGLUT1 (1:200, Merck, AMAB91041), rat monoclonal anti-Dopamine Transporter, DAT (1:200, Abcam, ab5990), rabbit anti-VGAT (1:200, Merck, AB5062P), mouse anti-GFAP (1:200, Merck, G3893), chicken polyclonal anti-MAP2 (1:200, Abcam, ab75713), rabbit anti-OLIG2 (1:200, Merck, HPA003254). Fluorescence images of sections were obtained using a widefield epifluorescence microscope (Leica DMi8) equipped with a 10×/0.32 HC PL Fluotar or a 20×/0.4 HC PL Fluotar objective lens.

### Ca²⁺ Imaging.

Live calcium imaging was performed with the same widefield epifluorescence microscope, equipped with temperature, CO₂, and humidity control. The tissues were incubated in the recommended concentrations of Fluo-4 direct according to the manufacturer (Molecular Probes, F10471) for 50 min in the incubator at 37° C. and 10 min at room temperature. After that, the tissues were washed five times with the neural maintenance medium. The calcium surges were recorded using an excitation of 488 nm and an emission of 530 nm every 10 seconds. Fluorescence images were obtained using a widefield epifluorescence microscope (Leica DMi8) equipped with either a 5×/0.15 HC PL Fluotar or a 10×/0.32 HC PL Fluotar objective lens.

### Penicillin Treatment.

After 15 minutes of calcium imaging in normal conditions, the chip was taken out of the live-imaging setup and 170 μl of the total volume of the treated chamber was replaced by a solution of Penicillin G sodium salt (Sigma, 13752) with a concentration of 100 mg/ml (equivalent to 2,8×10⁴ IU). The chip was quickly placed back in the same position and the live calcium imaging was continued. This process took approximately 3 min.

### Analysis of Network Interconnectivity.

Neuronal activity was analyzed from the time-lapse images. To account for possible drifts or deformations of the tissues, the location of the cells was detected in each frame and linked across the frames using the Mosaic particle tracker plugin in ImageJ. The fluorescence intensity of each detected cell was calculated from the image intensity data using MATLAB. Occasional gaps in the time traces, due to the cells not being detected in certain frames, were filled using spline interpolation of the intensity values. The background fluorescence in each chamber was calculated in the same way using 10 arbitrarily selected cell-sized ROIs in the cell-free regions of each chamber, averaged, and subtracted from the real (cell) data. Further, the intensity values were corrected for imaging artifacts due to out-of-focus fluorescence by subtracting the mean intensity of an annular mask with an outer radius of 18 pixels and an inner radius of 9 pixels (i.e., size of the ROI) for each ROI.

Further analysis of the functional interconnectivity between neurons was performed using a custom-written script in MATLAB (version R2018b, The Mathworks Inc.). The normalized rate of change in fluorescence (ΔF/F) was calculated using (F_{cell}−Fₘᵢₙ)/Fₘᵢₙ where F_{cell} is the mean fluorescence of a selected ROI measured in each frame and Fₘᵢₙ the lowest measured mean fluorescence value of that ROI throughout the imaging window. For the analysis of the neuronal activity upon Penicillin treatment, ΔF/F was calculated as (F_{cell}−Fᵢₙᵢₜ)/Fᵢₙᵢₜ, where Fᵢₙᵢₜ is the fluorescence intensity of the ROI at the beginning of the live imaging, to capture the jump in fluorescence intensity due to addition of Penicillin. Transient spikes with minimum peak prominence larger than twice the magnitude of stochastic noise in the cell-free regions were detected using the ‘findpeaks’ function. To identify synchronized neuronal firings, the Pearson's linear correlation coefficient r was computed for each pair of detected ROI. Following Eguiluz et al¹¹, we defined two ROIs to be functionally connected when r>0.6. To further assess the network modularity, we analyzed the functional connectivity using iterative Louvain community-detection algorithm^{19,20} with weighted edges to find the optimal network partitioning. Visualization of the network connectivity was realized using the graph plotting functions in MATLAB.

Diffusion rate measurements in the iS3CC device for analysis of relative diffusion rate, one of the chambers of the iS3CC device was loaded with 100 mg/ml of fluorescein sodium salt in Milli-Q water and the other chamber with pure Milli-Q. At different time points, samples of 5 μl were taken out from the pure Milli-Q chamber and added to a 96 well microplate to a final volume of 100 μl and fluorescent intensity was measured using a plate reader (Synergy HT). Based on calibration experiments, the final normalized concentrations in the MilliQ chamber indicated the relative diffusion rate over time.

### Results

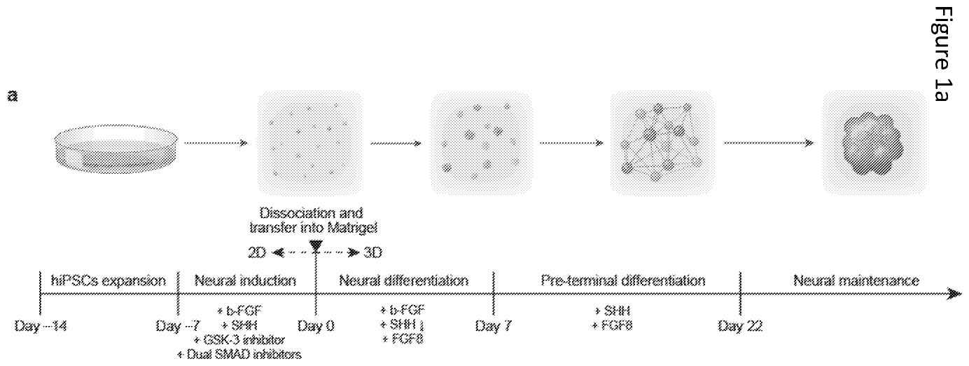

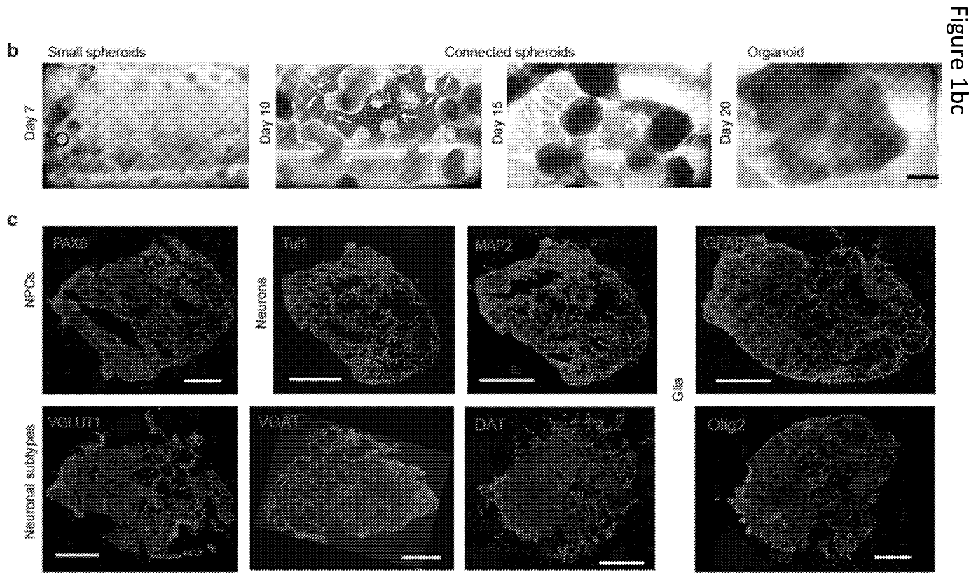

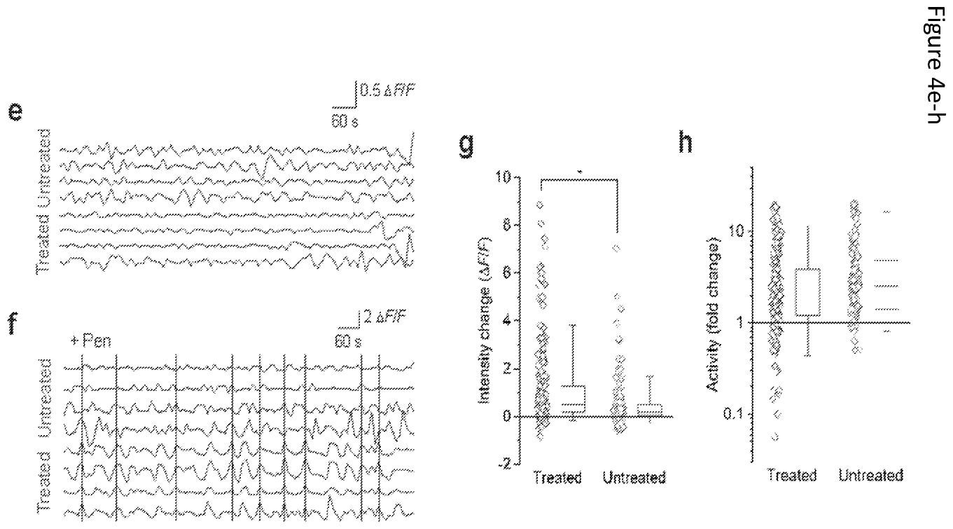

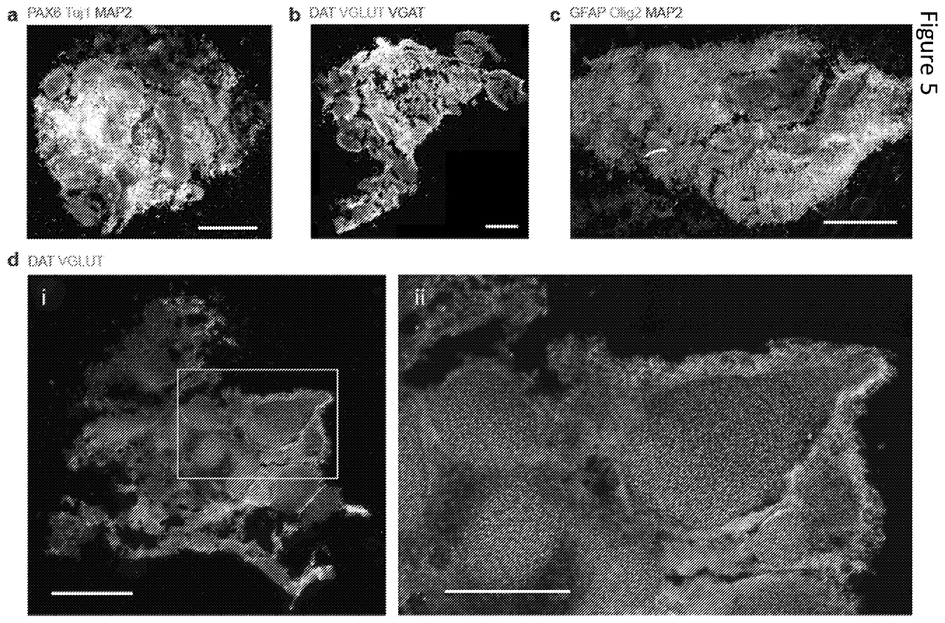

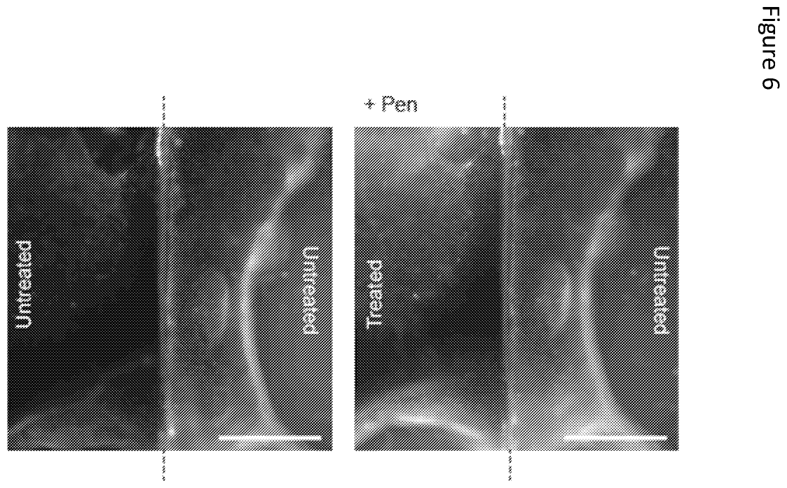

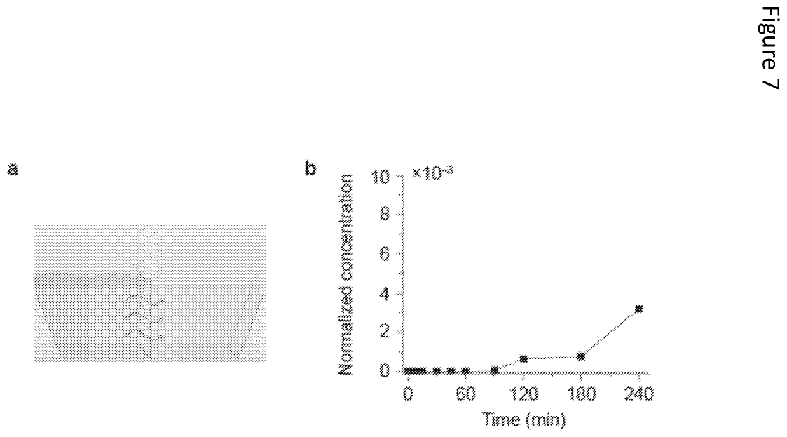

From a developmental perspective, one key step in the assembly of neuronal circuits and mature interconnected networks is neuronal migration⁸. This step is, however, inherently suppressed in the often used protocols to generate brain organoids (e.g., based on serum-free culture of embryoid body-like aggregates with quick reaggregation or SFEBq method⁷). In order to recapitulate this process, we introduce a novel culturing method to develop human cerebral tissue at the mesoscale (mm size) via matrix-supported active (migrative) reaggregation of cells (MARC). Unlike other protocols, e.g. reported for brain organoids, that use a quick, mechanically-enforced aggregation of the dissociated cells, the formation of the three-dimensional (3D) tissue (hereinafter referred to as “cerebral tissue”) is initiated by active (migrative) reaggregation of the cells during chemically-induced differentiation, with the immediate 3D extracellular support of Matrigel (FIG. 1a; see also experimental details in Methods). In order to promote active neurite-mediated reaggregation and engineer multiregional cerebral tissues, we rationally designed a neuronal differentiation protocol employing phased introduction and withdrawal of the culture additives used for neuronal tissue patterning (Bejoy et al 2016). After ˜80% confluence of the culture of human induced pluripotent stem cells (hiPSCs) under feeder-free condition, neuronal differentiation was initiated by addition of SMAD inhibitors (dorsomorphin and SB431542), a GSK-3 inhibitor (CHIR99021), SHH, and b-FGF. After 7 days of induction, the cells were enzymatically dissociated and homogenously resuspended in a mixture of Matrigel and neural differentiation medium containing b-FGF, SHH and FGF8. This step is henceforth considered Day 0 of MARC culture. Neural differentiation of 7 days therefore took place in a 3D environment, accompanied by the rapid formation of spheroids with a size of 200-300 μm (FIG. 1b, Day 7). Pre-terminal differentiation was started by removal of b-FGF from the medium, resulting in the formation of neurite outgrowth and bundles that connected the spheroids to each other (FIG. 1b, arrows and arrowheads). These spheroids merged over time (approximately 2 weeks), likely through a synapse-mediated migration 9, resulting in large cerebral tissues with a size of 2-4 millimeters (FIG. 1b, Day 15, 20). Finally, SHH and FGF8 were withdrawn and the cultures were treated with the maintenance medium. The cerebral tissues continued growing during the culture period of 90 days and expressed markers of distinct cell types of the human brain, confirming multiregional tissue patterning of our engineered cerebral tissues (FIG. 1c and FIG. 5). In addition, colocalized expression of different markers suggested spatial patterning of these cell populations (FIG. 5).

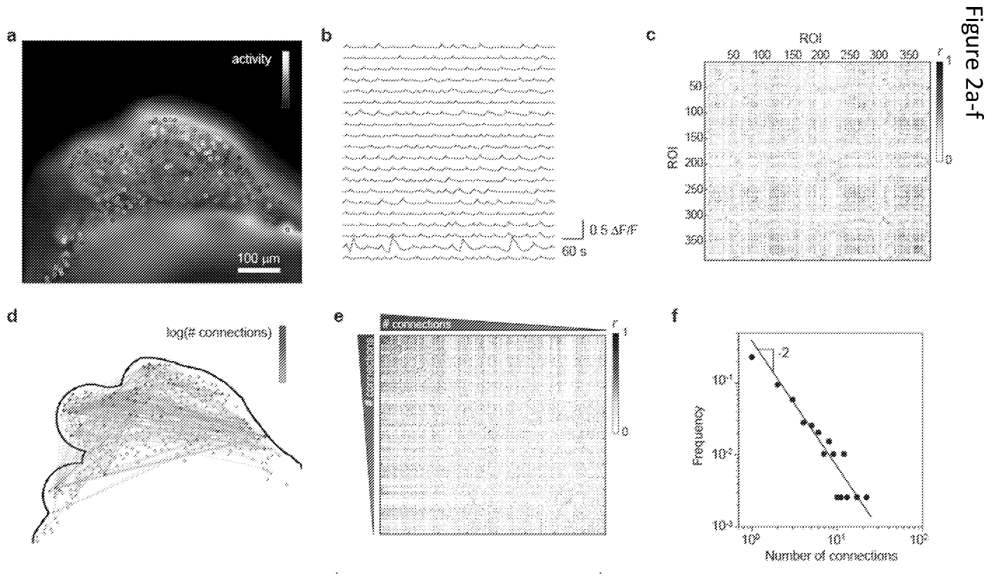

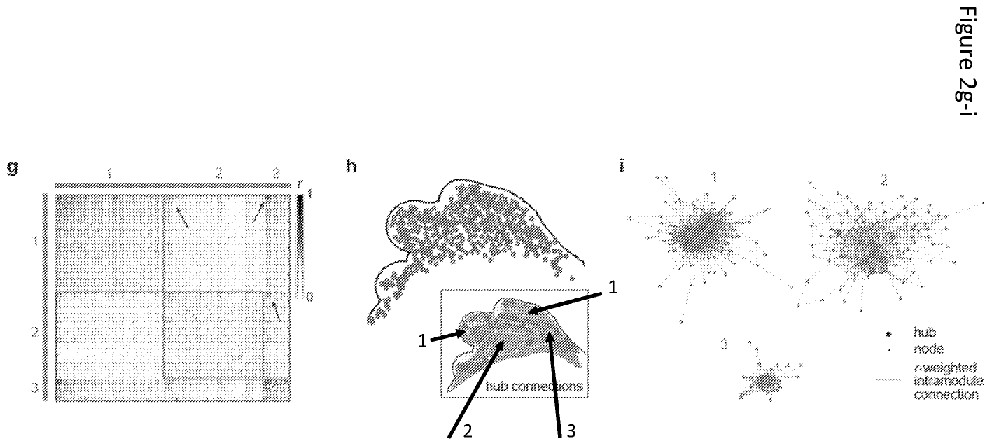

To test the neuronal functionality of MARC-produced cerebral tissues, we evaluated the neuronal interconnectivity within the intact 3D tissues using live calcium imaging. We observed that cerebral tissues at age 4 weeks exhibited extensive spontaneous calcium surges throughout the tissues (FIG. 2a,b and Supplementary Movie 1). Examination of the time-lapse images also indicated extensive synchronized neuronal firing, which is known to result from concentrated bursts of action potentials between interconnected neurons, leading to influx of extracellular calcium¹⁰. To quantify this population-wide intercellular synchronized activity, we computed the pairwise linear correlation coefficient r from the intensity time-trace of 387 regions-of-interest (ROIs) representing detected single neurons in the cerebral tissues. The correlation matrix between all ROI pairs shows that the majority of the ROI pairs had low r-values, but a significant number of pairs was highly correlated (FIG. 2c). We defined two ROIs to be functionally connected when r>0.6, following Eguiluz et al¹¹. Functional neuronal connection was found for 304 pairs (˜0.4% of all ROI pairs analyzed) and depicted in a spatial connection map (FIG. 2d), demonstrating a functional neuronal network. Interestingly, the functional connections were not randomly distributed throughout the cerebral tissues; several ROIs were highly connected to many other ROIs whereas most others only had a few connections. In fact, we found an increased synchrony between the nodes with higher amount of connections (FIG. 2e). These observations are consistent with the topological features of scale-free networks, which have been proposed to be important for synchronized functional networks¹²⁻¹⁴. To check whether the functional interconnectivity in the cerebral tissues follows a scale-free topology, we plotted the distribution of the number of connections each neuron has. Indeed, the distribution follows a power law with a decay constant of −2 (FIG. 2f), consistent with the characteristics of scale-free network¹⁵ and in agreement with clinical measurement of whole-brain activity¹⁶. The presence of a small number of hyper-connected “hub-like” cells (up to >20 connections in our case) and a large number of cells with few connections results in a low average number of connections (˜1.5 in our cerebral tissues). It has been proposed that brain achieves large-scale interconnectivity between brain regions despite the low average number of connection through a modular network topology, whereby the network is composed of subnetworks (“modules”) of densely interconnected neurons (“nodes”). Such modular architecture is thought to be critical for the emergence of adaptive behaviours and cognition^{17,18}. To assess the network modularity in our cerebral tissues, we analysed the functional connectivity using iterative Louvain community-detection algorithm^{19,20} (see Methods). The algorithm identified 3 distinct modules within the tissue with sparse intermodular node connections (FIG. 2g,h). Moreover, each module includes its own local hubs that are highly interconnected (inset in FIG. 2h), which ensure global integration of functional interconnections across the overall network²¹. Within each module, the nodes are interconnected in a hierarchical topology, from a few hub nodes with high number of connections that are closely connected to each other to peripheral nodes at the outer edges of the network topology (FIG. 2i). Taken together, the analysis demonstrates a rich interconnectivity in the cerebral tissues reminiscent of the emerging attributes of functional networks in brain, suggesting their utility as an in vitro mimic of brain functional interconnectivity.

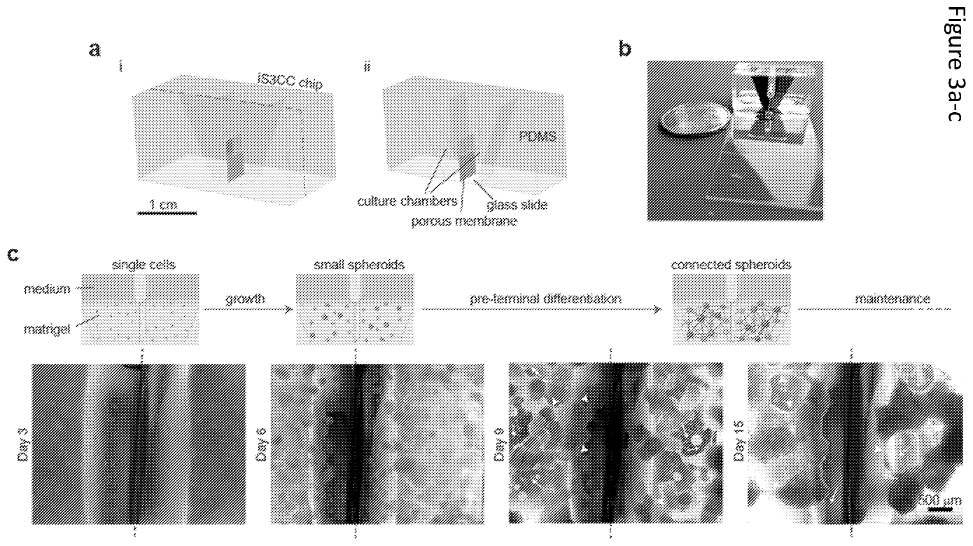

Having established the functional interconnectivity within the MARC-produced cerebral tissues, we wanted to study 3D interconnectivity and signal transmission between living, interconnected cerebral tissues. To this end, we designed and fabricated a polydimethylsiloxane (PDMS) chip with optimized features, consisting of two culture chambers that are individually accessible and separated by a porous membrane (FIG. 3a). The design and dimensions of the vertically-tapered chambers were chosen to suit one-pot formation of MARC-produced cerebral tissues and to maintain nutrition and oxygen supply, while at the same time allowing simultaneous visualization of both chambers in a side-by-side configuration through a glass slide at the bottom (FIG. 3b). The porous membrane separating the chambers was chosen to have pore sizes of 8 μm to keep cerebral tissues separated yet allow spontaneous neurite interconnection across the membrane. Following the MARC culture protocol, we generated cerebral tissues in each of the two chambers, which formed in a similar way as described earlier, including the neurite-assisted spheroid reaggregation into cerebral tissues (FIG. 3c). Importantly, the tissues were observed to spontaneously connect with each other through the porous membranes (FIG. 3d). Live calcium imaging showed frequent calcium surges across the membrane (FIG. 3e and Supplementary Movie 2), indicating the presence of functional interconnectivity between the in the two chambers. Therefore, the combination of cerebral tissue formation using MARC and this interacting separated 3D co-culture (iS3CC) chip is ideally suited for investigations into the signal transmission between interconnected cerebral tissue cultures.

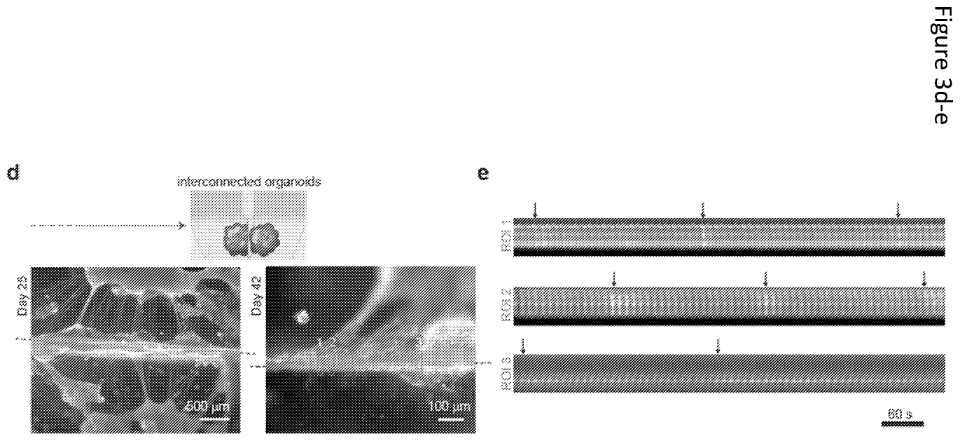

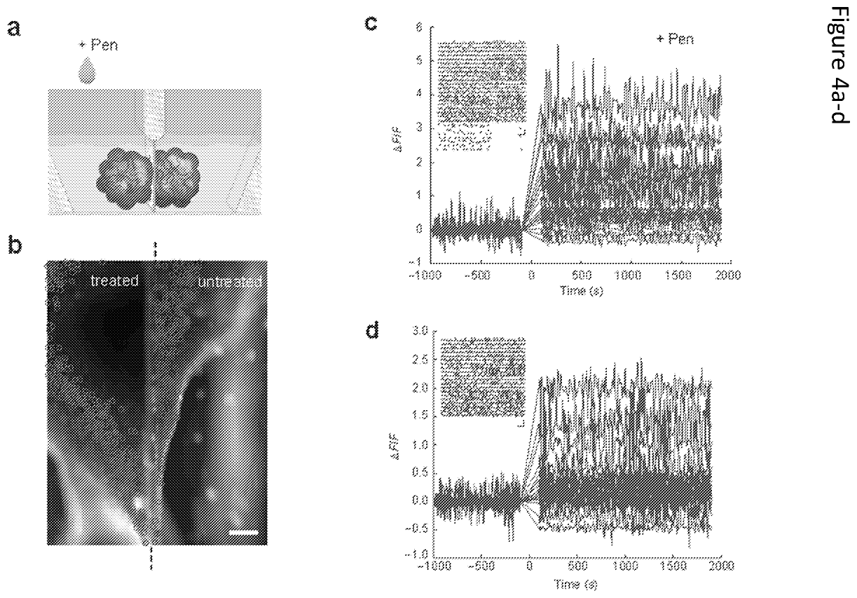

Finally, as a proof of concept of our approach, we sought to demonstrate its utility to mimic a neurological disorder affecting network interconnectivity in a controlled in vitro environment. Epilepsy is a chronic network-level disease defined and diagnosed by the occurrence of one or more unprovoked epileptic seizures, which are caused by alterations in the brain network circuits and functional interconnectivity. Physiologically, epileptic seizures are characterized by a transient occurrence of abnormal excessive or synchronous neuronal activity and spatial propagation of these abnormal activities²³. To experimentally induce epileptic seizures, we used the neurotoxic properties of Penicillin G²⁴, a γ-aminobutyric acid (GABA) A-receptor (GABA_{A}R) blocker of the β-lactam antibiotics family²⁵. The epileptiform mechanism of Penicillin G has been theoretically and experimentally shown to occur through the specific binding of Penicillin G with GABA_{A}R in an open configuration, which prevents GABAergic transmission in CNS²⁶ and results in a hypersynchronous activity in the brain due to interference of the GABA-inhibition and glutamate-excitation equilibrium, causing abnormal electrical discharges²⁷. Here we intended to simulate propagation of abnormal discharges from one cerebral tissue to another. Therefore, we formed interconnected cerebral tissues in the two chambers of the iS3CC chip, treated one chamber with Penicillin G by bath application to the culture medium (FIG. 4a), and monitored the neuronal activity in both chambers using live calcium imaging (FIG. 4b,c). Prior to the Penicillin G treatment, both cerebral tissues showed comparable level of neuronal activities (FIG. 4b,c inset). Immediately following the Penicillin G treatment, we observed an increased amount of fluorescence in the treated tissue, compared to the baseline (FIG. 4b,g, Figure S2, and Supplementary Movie 3). This is accompanied by an increased magnitude and frequency of neuronal activity, as well as synchrony of transient spikes in the treated tissue (FIG. 4e,f,h, in blue), indicative for abnormal excessive discharges. Subsequently, the neurons in the untreated chamber similarly showed an increased intensity and neuronal activity post-treatment (FIG. 4c,g), despite negligible inter-chamber particle diffusion (Figure S3). This observation strongly suggests that these immediate changes in the untreated tissue result from discharge propagation through inter-tissue connections across the membrane. To our knowledge, this is the first time that signal transmission of abnormal activities (i.e., epileptiform discharge propagation) has been recapitulated in vitro.

One of the key elements of the development of nervous systems is the formation of complex neuronal networks^{28,29}. The engineered cerebral tissues in this study showed characteristics of mature neuronal networks, including synchronized influxes of extracellular calcium and modular functional connectivity patterns, demonstrating the formation of interconnected network within the intact tissues. Indeed, our culture method of promoting active reaggregation of cells and spheroids minimizes exogenous (mechanical) perturbations that may lead to cellular stress and unknown effects on tissue functionality³⁰. As such, the MARC-produced cerebral tissues can be used to study physiological and pathophysiological features of healthy and diseased neuronal networks. In general, neurological disorders are known to be accompanied by alterations in the network and functional interconnectivity between different brain regions³¹⁻³³. Examples of these disorders include epilepsy, Alzheimer's disease, schizophrenia, multiple sclerosis, depression, ASD, and traumatic brain injury. To further test the ability of our cerebral tissues to serve as a model of network interconnectivity disorders, we induced abnormal excessive discharges in the tissue cultured in one chamber of the iS3CC chip, which was found to lead to increased neuronal activity in the untreated tissue. These data indicate propagation of excessive discharges, which is the underlying mechanism of occurrence of an epileptic seizure and the target of most of current anti-epileptic drug treatments. Moreover, our study demonstrates a novel approach to develop and analyze interconnected brain tissue that opens a wide range of possibilities to mechanistically study clinically relevant interregional functional connectivity in lab grown 3D brain tissues.

### REFERENCES

1. Quadrato, G. et al. Cell diversity and network dynamics in photosensitive human brain organoids. Nature 545, 48-53 (2017).
2. Giandomenico, S. L. et al. Cerebral organoids at the air-liquid interface generate diverse nerve tracts with functional output. Nat. Neurosci. 22, 669-679 (2019).
3. Birey, F. et al. Assembly of functionally integrated human forebrain spheroids. Nature 545, 54-59 (2017).
4. Paşca, A. M. et al. Functional cortical neurons and astrocytes from human pluripotent stem cells in 3D culture. Nat. Protoc. 12, 671-678 (2015).
5. Lancaster, M. A. et al. Guided self-organization and cortical plate formation in human brain organoids. Nat. Biotechnol. 35, 659-666 (2017).
6. Lancaster, M. A. & Knoblich, J. A. Organogenesis in a dish: Modeling development and disease using organoid technologies. Science 345, 1247125. (2014).
7. Eiraku, M. et al. Article Self-Organized Formation of Polarized Cortical Tissues from ESCs and Its Active Manipulation by Extrinsic Signals. Stem Cell 3, 519-532 (2008).
8. Valdeolmillos, M. & Moya, F. Leading Process Dynamics During Neuronal Migration. in Cellular Migration and Formation of Neuronal Connections (eds. Rubenstein, J. L. R. & Pasko, R.) 245-260 (Academic Press, 2013). doi:10.1016/B978-0-12-397266-8.00025-9.
9. Ohtaka-Maruyama, C. et al. Synaptic transmission from subplate neurons controls radial migration of neocortical neurons. Science 360, 313-317 (2018).
10. Robinson, H. P. et al. Periodic synchronized bursting and intracellular calcium transients elicited by low magnesium in cultured cortical neurons. J. Neurophysiol. 70, 1606-1616 (1993).
11. Eguiluz, V. M., Chialvo, D. R., Cecchi, G. A., Baliki, M. & Apkarian, A. V. Scale-Free Brain Functional Networks. Phys. Rev. Lett. 94, 018102 (2005).
12. Feldt, S., Bonifazi, P. & Cossart, R. Dissecting functional connectivity of neuronal microcircuits: experimental and theoretical insights. Trends Neurosci. 34, 225-236 (2011).
13. Batista, C. A. S., Batista, A. M., Pontes, J. A. C. De, Viana, R. L. & Lopes, S. R. Chaotic phase synchronization in scale-free networks of bursting neurons. Phys. Rev. E 76, 016218 (2007).
14. Grinstein, G. & Linsker, R. Synchronous neural activity in scale-free network models versus random network models. Proc. Natl. Acad. Sci. 102, 9948-9953 (2005).
15. Barabási, A. & Reka, A. Emergence of Scaling in Random Networks. Science 286, 509-512 (1999).
16. He, B. J. Scale-free brain activity: past, present and future. Trends Cogn Sci 18, 480-487 (2015).
17. Mcintosh, A. R. Towards a network theory of cognition. Neural Networks 13, 861-870 (2000).
18. Cohen, J. R. & Esposito, M. D. The Segregation and Integration of Distinct Brain Networks and Their Relationship to Cognition. J. Neurosci. 36, 12083-12094 (2016).
19. Blondel, V. D., Guillaume, J., Lambiotte, R. & Lefebvre, E. Fast unfolding of communities in large networks. J. Stat. Mech. theory Exp. 10, P10008 (2008).
20. Pedersen, M., Zalesky, A., Omidvarnia, A. & Jackson, G. D. Multilayer network switching rate predicts brain performance. Proc. Natl. Acad. Sci. 115, 13376-13381 (2018).
21. Heuvel, M. P. Van Den, Stam, C. J., Kahn, S. & Pol, H. E. H. Efficiency of Functional Brain Networks and Intellectual Performance. J. Neurosci. 29, 7619-7624 (2009).
22. Fruchterman, T. M. J. & Reingold, E. M. Graph Drawing by Force-directed Placement. Software-practice Exp. 21, 1129-1164 (1991).
23. Fisher, R. S. et al. Operational classification of seizure types by the International League Against Epilepsy: position paper of the ILAE Commission for Classification and Terminology. Zeitschrift fur Epileptol. 58, 522-530 (2017).
24. Johnson, H. C. & Walker, E. Intraventricular penicillin: a note of warning. J. Am. Med. Assoc. 127, 217-219 (1945).
25. Avoli, M. & Jefferys, J. G. R. Models of drug-induced epileptiform synchronization in vitro. J. Neurosci. Methods 260, 26-32 (2016).
26. Rossokhin, A. V., Sharonova, I. N., Bukanova, J. V., Kolbaev, S. N. & Skrebitsky, V. G. Block of GABAA receptor ion channel by penicillin: Electrophysiological and modeling insights toward the mechanism. Mol. Cell. Neurosci. 63, 72-82 (2014).
27. Treiman, D. M. GABAergic mechanisms in epilepsy. Epilepsia 42, 8-12 (2001).
28. Katz, L. C. & Shatz, C. J. Synaptic Activity and the ConstructKatz, L C; Shatz, CJion of Cortical Circuits. Science 274, 1133-1138 (1996).
29. Zhang, L. I. & Poo, M. M. Electrical activity of neural circuits. Nat. Neurosci. 4, 1207-1214 (2001).
30. Bhaduri, A. et al. Cell stress in cortical organoids impairs molecular subtype specification. Nature 578, 142-148 (2020).
31. Soliman, M. A., Aboharb, F., Zeltner, N. & Studer, L. Pluripotent stem cells in neuropsychiatric disorders. Mol. Psychiatry 22, 1241-1249 (2017).
32. Nobili, A. et al. Dopamine neuronal loss contributes to memory and reward dysfunction in a model of Alzheimer's disease Annalisa. Nat. Commun. 8, 1-14 (2017).
33. Lubrini, G., Martin-Montes, A., Diez-Ascaso, O. & Diez-Tejedor, E. Brain disease, connectivity, plasticity and cognitive therapy: A neurological view of mental disorders. Neurologia 33, 187-191 (2018).

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding patent applications, patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

1. (canceled)

2. An in vitro method of producing a three dimensional neural tissue composition, the method comprising the steps of
a) providing pluripotent stem cells;
b) optionally, culturing the pluripotent stem cells in the presence of at least one neural induction medium;
c) re-suspending the cells of step a) or b) in a cell culture substrate, preferably wherein the re-suspended cells are dispersed in the cell culture substrate;
d) inducing re-aggregation and/or differentiation of the cells that are resuspended in the cell culture substrate, preferably by culturing the cells that are resuspended in the cell culture substrate in the presence of at least one neural differentiation medium or in the presence of at least one neural induction medium followed by culturing in the presence of at least one neural differentiation medium;
e) optionally, culturing the cells of step d) in the presence of at least one neural maintenance medium.

3. The in vitro method according to claim 2 wherein the pluripotent stem cells are human pluripotent stem cells, non-human pluripotent stem cells, human induced pluripotent stem cells, non-human induced pluripotent stem cells, human embryonic stem cells, non-human embryonic stem cells, human non-embryonic stem cells, or non-human non-embryonic stem cells.

4. The in vitro method according to claim 2 wherein the pluripotent stem cells are cultured, preferably until at least 60-80% confluence, in the presence of a pluripotent stem cell proliferation medium before culturing the cells in the presence of the at least one neural induction medium.

5. The in vitro method according to claim 2 wherein the neural induction medium comprises:
a) at least one compound that inhibits Small Mothers Against Decapentaplegic (SMAD) protein signaling (“SMAD inhibitor”), preferably wherein said at least one SMAD inhibitor is selected from the group consisting of dorsomorphin, SB431542, noggin, LDB193189, or any combination thereof, even more preferably wherein said at least one SMAD inhibitor comprises dorsomorphin and SB431542;
b) at least one compound that activates Wnt-signaling, preferably wherein said compound inhibits Glycogen synthase kinase 3 (“GSK-3 inhibitor”), preferably wherein said GSK-3 inhibitor is selected from the group consisting of CHIR99021, CHIR98014, and 6-bromoindirubin-3′-oxime;
c) at least one compound that activates Sonic Hedgehog signaling (“SHH activator”), preferably wherein said SSH activator is selected from the group consisting of a SSH protein, pumorphamine, SAG smoothened agonist, and Hh-Ag1.5; and/or
d) basic Fibroblast Growth Factor (“b-FGF”).

6. The in vitro method according to claim 2 wherein the at least one neural induction medium comprises at least one SMAD inhibitor, at least one GSK-3 inhibitor, at least one SHH activator, and bFGF, preferably wherein the neural induction medium comprises dorsomorphin, SB431542, CHIR99021, SHH, and b-FGF.

7. The in vitro method according to claim 2 wherein culturing the pluripotent stem cells in the presence of at least one neural induction medium is for a period of at least 2, 3, 4, or 5 days, preferably between 2-15 days, 3-10 days or 4-9 days.

8. The in vitro method according to claim 2 wherein culturing the pluripotent stem cells in the presence of at least one neural induction medium is 2D culturing.

9. The in vitro method according to claim 2 wherein the cell culture substrate comprises extracellular matrix components and/or wherein the cell culture substrate comprises Matrigel, gelatin, vitronectin, laminin, fibronectin, and/or collagen, preferably the cell culture substrate is Matrigel.

10. The in vitro method according to claim 2 wherein the pluripotent stem cells, or the cells obtained after culturing of induced pluripotent stem cells in the neural induction medium, are obtained, preferably by preparing a cell suspension, and resuspended in the cell culture substrate, preferably wherein the re-suspended cells are dispersed in the cell culture substrate, preferably wherein the cell culture substrate comprises extracellular matrix components and/or wherein the cell culture substrate comprises Matrigel, gelatin, vitronectin, laminin, fibronectin, and/or collagen, preferably the cell culture substrate is Matrigel.

11. The in vitro method according to claim 2 wherein culturing the resuspended cells in the presence of at least one neural differentiation medium in step d) comprises
i) culturing the resuspended cells in the presence of a first neural differentiation medium;
ii) culturing the resuspended cells in the presence of a second neural differentiation medium;
wherein said first, and second neural differentiation medium each have a different composition, preferably wherein:
the first neural differentiation medium comprises b-FGF, at least one SHH activator, preferably SSH protein, and a Fibroblast growth factor 8 protein (“FGF8”); and
the second neural differentiation medium comprises at least one SHH activator, preferably SSH protein, and a Fibroblast growth factor 8 protein (“FGF8”), and is substantially free of b-FGF.

12. The in vitro method according to claim 2 wherein culturing the resuspended cells in the presence of at least one neural differentiation medium is for a period of at least 5, 6, or 7 days, preferably between 5-45 days, 8-35 days or 10-25 days.

13. The in vitro method according to claim 2 wherein culturing the resuspended cells in the presence of the first neural differentiation medium is for a period that is shorter than the period for culturing in the presence of the second neural differentiation medium, preferably wherein culturing in the presence of the first neural differentiation medium is for a period between 1 and 10 days and/or wherein culturing in the presence of the second neural differentiation medium is for a period of between 5 and 30 days.

14. The in vitro method according to claim 2 wherein the at least one neural maintenance medium is substantially free of an SHH activator, preferably SSH protein, a Fibroblast growth factor 8 protein (“FGF8”) and b-FGF.

15. The in vitro method according to claim 2 wherein the cells are cultured in the presence of at least one neural maintenance medium for a period of at least 10, 15, 25, 40, 80 or 90 days.

16. A composition comprising three dimensional neural tissue composition obtained with the method according to claim 2.

17. (canceled)

18. (canceled)
